(19) United States
(12) Patent Application Publication
(10) Pub. No.: US 2004/0253735 A1
(43) Pub. Date: Dec. 16, 2004

(54) Method for measuring liquid hydrocarbons using refractive index

(75) Inventors: Kiran Vadgama, Calgary (CA); Roger Chetwynd, Calgary (CA)

Correspondence Address: BENNETT JONES, C/O MS ROSEANN CALDWELL, 4500 BANKERS HALL EAST, 855 - 2ND STREET, SW, CALGARY, AB, T2P 4K7, CA

(73) Assignee: OCTAL SYSTEMS INCORPORATED

(21) Appl. No.: 10/687,661

(22) Filed: Oct. 20, 2003

(30) Foreign Application Priority Data
Jun. 13, 2003 (CA) 2,432,403

(51) Int. Cl. G01N 33/18

(52) U.S. Cl. 436/40

(57) ABSTRACT

The present invention is an apparatus and method for measuring the content of water in unrefined hydrocarbons through the application of a refractive index comprising a capacitance measuring device and a density measuring device. The capacitance measuring device may be calibrated according to linear data regarding the refractive index of an emulsion of oil and water to provide output. The output of the capacitance device may be used to obtain a refractive index of the emulsion. The density measuring device may be used to measure the density of the emulsion from which the actual density of oil may be obtained. The density oil may then be applied to a linear curve to find the value of the refractive index of the oil and from this measurement the water content of the emulsion may be obtained.

### FIELD OF THE INVENTION

The present invention relates to an apparatus and method for measuring the content of water in unrefined hydrocarbons such as an emulsion of water and oil.

### BACKGROUND OF THE INVENTION

The process industries including chemical, pharmaceutical, and oil and gas industries, use numerous analytical techniques for research, quality control, production and accounting purposes. In the oil and gas industry many types of oils and emulsions and other materials can be characterized through the measurement and co-relation of a number of different fundamental properties such as temperature, pressure, density, dielectric, conductivity, refractive index, viscosity, elemental composition, and other properties.

Hydrocarbon processing plants, transmission pipelines, and many other facilities require quantitative monitoring of fluid flow. In the oil and gas industry, some of the most critical applications of flow measurement are undertaken by companies involved in fiscal transactions that are associated with the transfer of hydrocarbon gas, or liquid, from a seller to a buyer. The measurement of oil and water flow is one specific instance that is often used as a basis for commerce between oil and gas producers, transporters and distributors.

The flowing conditions may fluctuate throughout the duration of the measurement of fluid transfers due to varying ambient and process conditions. Most petroleum fluids are measured and sold on a volumetric basis. Due to the fact that the density of all these fluids varies with pressure, temperature and water content, the measured volume at one set of process conditions may be significantly different from another set of process conditions. To obtain consistent and accurate dynamic flow measurements, no matter what the ambient conditions may be, the flow measurement devices must convert the measured volume to an equivalent volume referenced to a set of standard conditions acceptable to both parties. In Canada and the US, the petroleum industry and the corresponding regulatory bodies have embraced the Measurement and Data Management standards published by the American Petroleum Institute (API), the American Gas Association (AGA) and the Gas Processing Association (GPA). In Canada, the custody transfer (CT) process must also comply with some very specific requirements laid out by Industry Canada.

One such requirement, for example, applies to accurately measuring the net oil volume delivered in tanker trucks to an oil battery for processing. There are many truck terminal facilities in oil and gas production areas in the world where trucks off-load crude oil, or lighter condensate, hauled from various production wells. Each load that is off-loaded is measured by an online flow computer which, based on the density and ambient conditions of the product being off-loaded, corrects the measured volume to reference conditions. The value of the referenced volume is a function of the flowing density, flowing temperature and flowing pressure and of course water content. For example, when a batch of oil and water emulsion is off-loaded at a truck terminal facility, the facility operators have to decide whether the water content is lower than the accepted limit which is usually around 0.5% water or less. Based on its water content, the hydrocarbon fluid being off-loaded from the truck may be transferred to a clean oil tank or an emulsion tank. Sometimes the facility may reject the product if it does not meet the required minimum purity level. Any method or apparatus that can provide this information on-line can make the operation of the facility more economical and profitable.

There are several devices and processes in use today that facilitate the determination of water content of unrefined oil and water mixtures. One class of such devices is the capacitance probe that measures the capacitance between two plates or concentric cylinders when oil or an emulsion is flowing therethrough. In general such devices can measure water fraction to an accuracy margin of 0.1% or better, under narrowly defined conditions of calibration. Almost all of these devices use dielectric constant as measured capacitance to calculate one fundamental property of the hydrocarbon fluid containing water passing through the plates. The calculated dielectric value is then used in conjunction with measurements of fluid density and temperature to calculate the water content. In almost every case, the significant difference between the value of the dielectric constant of the hydrocarbon fluid and the value for the dielectric constant of water serves as a basis for measuring the amount of water present in the hydrocarbon fluid.

There are several variations in the way the dielectric constant, the temperature and the density of oil, water and emulsion may be utilized to determine the water content in a hydrocarbon fluid. Some apparatus and methods provide water content of hydrocarbons at a single calibrated density and temperature. Other apparatus and methods allow for some temperature variation by applying some form of mathematical or empirical correction with reference to one calibration point.

One application in particular provides water content for wider flowing density variations by generating nonlinear empirical data for dielectric constants versus density from a finite number of density values. This method can be a cumbersome and a time consuming process. In such a method the process may be calibrated to correctly read water content in oil at one specific oil density. A batch of an oil and water mixture with a mid-range density may be used as a convenient calibration point. Due to the nonlinear empirical nature of the process, it can be virtually impossible to apply a composite calibration procedure to a probe that covers a broader range of densities. Therefore, such an application may provide acceptable results for a given batch of an oil and water mix for which it has been calibrated. However, a correction or bias factor is applied to all other batches that deviate from the calibration density. This can be an extremely cumbersome process and to some extent negates the advantage of continuous online density and dielectric measurement to calculate the water content of the oil/water mixture; it can be akin to calibrating the process for every expected batch density. Storage in the computing module of such correction/calibration factors associated with a given batch can make this procedure possible.

Another application has attempted to overcome the non-linearity of the dielectric constant versus oil density by using the square root of the dielectric constant and generating a linear plot. Such an application may not overcome the problem inherent in single point calibration. Such an application proposes to use a straight line relationship with its intercept for zero density at a dielectric value of 1. The application of such an assumption may produce erroneous results since the instrument or measurement device may be affected by the physical installation of the measurement probe not only at the zero intercept point but also at the upper full scale operating point.

The effect of temperature on the density of oil has been well established and has been documented in published industrial standards over many years. As the temperature of oil increases, its density decreases. It is well known that the dielectric constant of oil varies with density. It also varies with temperature because the density varies with temperature. Due to the effect of variables upon the accuracy of the dielectric constant, an apparatus and means of establishing the water content of an emulsion that does not utilize a dielectric constant may be more accurate than the existing systems.

### SUMMARY OF THE INVENTION

In accordance with a broad aspect of the present invention, there is provided an apparatus for measuring the content of water in an emulsion including hydrocarbons and water, the apparatus comprising: a conduit through which the emulsion may flow from a first point to a second point; at least one measurement device selected to measure density and capacitance of the emulsion within the conduit to generate a density value and a capacitance value; and a computing device capable of receiving the density value and the capacitance value from the at least one measurement device and for the purpose of determining the content of water in the emulsion through the application of a refractive index.

In accordance with a broad aspect of the present invention, there is provided a method for measuring the content of water in an emulsion including hydrocarbons and water, the method comprising: providing at least one measurement device for obtaining a density value and a capacitance value from the emulsion; providing a computing device capable of receiving the density value and the capacitance value from the at least one measurement device; and using the density value and the capacitance value to determine: a refractive index value of the emulsion; at least one calibration value; and at least one water content value of the emulsion.

In accordance with a broad aspect of the present invention, there is provided a method for measuring the content of water in an emulsion including hydrocarbons and water, the method comprising: obtaining a density value and a capacitance value from the emulsion; using the density value and the capacitance value to determine: a refractive index value of the emulsion; at least one device calibration value; and at least one water content value of the emulsion.

These and other features and advantages of the apparatus and method for measuring the content of water in unrefined hydrocarbons according to the present invention will become more apparent with reference to the following detailed description and drawings.

### BRIEF DESCRIPTION OF THE DRAWINGS

FIG. 1 is a block diagram showing an apparatus of the invention.

FIG. 2 is a cross-section view of an emulsion between two capacitor plates.

FIG. 3 is a flow diagram of a process calculation.

FIG. 4 is a flow diagram of a calibration process.

FIG. 5 is a graph of data for refined oils.

FIG. 6 is a list of process and other variables used for calibration.

FIG. 7 is a graph of refractive index versus measured capacitance for batch 1.

FIG. 8 is a graph of refractive index versus measured capacitance for batch 2.

### DETAILED DESCRIPTION OF THE INVENTION

An apparatus and a method for measuring the content of water in a hydrocarbon emulsion with water is disclosed. The apparatus and method are selected to apply a refractive index generated from capacitance measurement and a density measurement.

It is advantageous to use a refractive index for the determination of a water content in an oil due to the fact that the relationship between the density of oil and its refractive index is linear. Therefore, in principle two measurements of oil density and its refractive index can be obtained to establish a linear mathematical relationship. This provides an accurate, reliable and repeatable water content determination over a wider range of oil densities, ease of calibration for water content determining apparatus and a calibration process which is independent of the physical dimensions of the capacitance measuring device.

In the apparatus, measurement devices access the emulsion as it flows through a conduit to derive density, temperature and capacitance values. These values are transferred to a computing system which may apply a series of equations to a determination of the water fraction existing in the emulsion. A refractive index value may also be determined by the computing device and applied to the derivation of the water fraction.

The capacitance measuring device may be calibrated according to linear data regarding the refractive index of an emulsion of oil and water to provide output. The output of the capacitance device may be used to obtain a refractive index of the emulsion. The density measuring device may be used to measure the density of the emulsion from which the actual density of oil may be obtained. The density oil may then be applied to a linear curve with the value of the refractive index of the oil and from this measurement the water content of the emulsion may be obtained.

In a method for measuring the content of water in an emulsion including hydrocarbons and water, the method includes obtaining a density value and a capacitance value from the emulsion and using the density value and the capacitance value to determine: a refractive index value of the emulsion; at least one device calibration value; and at least one water content value of the emulsion.

One embodiment involves an iterative process to derive values required to determine a water fraction value. This method of deriving values involves multiple determinations of a value. Each determination being compared to a second value. If the determined and the second value are not within a predetermined variable range then the iterative process may continue until a determined value within the range is derived. The application of an iterative process may allow for a more accurate water fraction value to be derived than non-iterative processes.

Another embodiment of the invention may determine two values for each of the density, temperature and capacitance measurements. On two separate instances the measuring device or devices, may measure the density, temperature and capacitance of the emulsion. The two sets of measured values can be used in conjunction with the iterative process as a further means of ensuring the accuracy of the determined water fraction value.

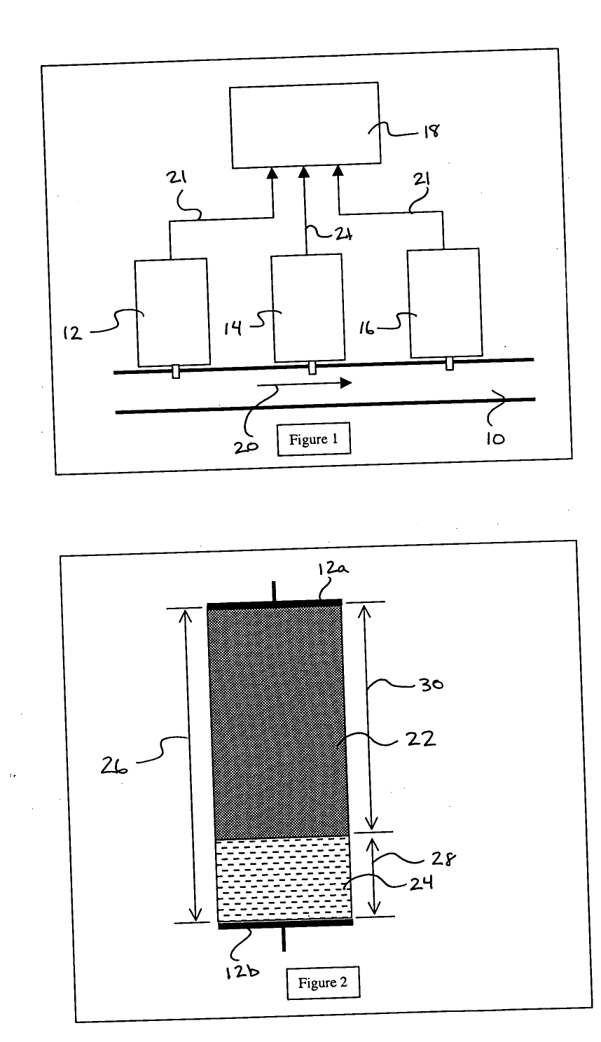

FIG. 1 shows an embodiment of the invention incorporating an apparatus that may be used to determine the content of water in a mixture of water and oil, or any other type of emulsion, as it flows through a conduit 10. The conduit 10 may be a pipe or another contained channel through which an emulsion may flow. One embodiment of the invention may connect the conduit 10 between a truck and a facility for the purpose of off-loading the emulsion. The conduit 10 may also be connected in other ways to convey an emulsion therethrough.

Some of the fluid flowing through the conduit 10 may reach the measurement devices represented by 12, 14 and 16. A capacitance measuring device 12 may provide a means of measuring the capacitance of the fluid flowing through the conduit 10. A density measuring device 14 may measure the density of the fluid flowing through the conduit 10. The temperature of the fluid flowing through the conduit 10 may be measured by a temperature measuring device 16. A person skilled in the art of measuring apparatus would recognize that a number of combinations and varieties of measuring devices may be used. For example, one or more devices can incorporate the various measurement systems and the systems can be of various configuration and types. It is to be understood that, if desired, the devices can include only density and capacitance measuring capabilities without referencing temperature. However, temperature is useful for enhancing the accuracy of the water fraction determinations.

Each measuring device 12, 14 and 16, is capable of producing a process variable signal to express the measurement value it derives, which the device may convert to a process variable signal. A computer 18, or another type of computing device, may be connected to the measurement devices 12, 14 and 16 and may monitor the values of the process variable signals provided by the measuring devices. The process variables may be used by the computer 18 to determine the fraction of water content existing in the fluid flowing through the conduit 10.

In the illustrated embodiment, the emulsion entering the conduit 10 from the direction of the arrow 20 may be monitored or measured by the capacitance 12, density 14 and temperature 16 devices as well as any other measuring device, as it flows. The process variable signals generated by the measuring devices may be a digital or an analog signal, or any other form of signal, that may be uploaded 21 to the computing device 18. Each device may generate a value, so that the capacitance device 12 may generate a capacitance value, the density device 14 may generate a density value and the temperature device 16 may generate a temperature value. Each generated value may be represented as a process variable signal and be uploaded 21 to the computing device 18. The signals, representing the measurement values derived by the measurement devices from the emulsion, may be used by the computing device 18 to determine the fraction of the content of the emulsion that is water.

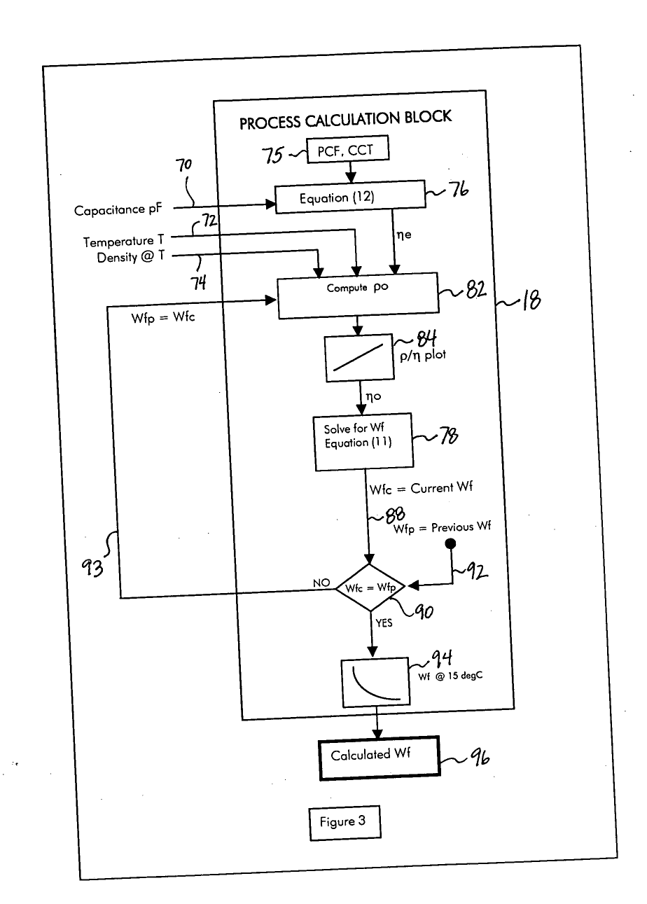

FIG. 2 shows a vertical column of an oil and water emulsion between two capacitor plates 12a and 12b within a capacitance device 12. In one embodiment of the invention electromagnetic radiation may propagate from one capacitor plate 12a to the other 12b as the oil and water emulsion flows between them. Due to the fact that oil and water do not mix, tiny emulsion “droplets” may exist in the fluid, and any given electromagnetic wave may pass through successive “droplets” of oil and water. The wave may therefore pass through a single large drop so that it passes first through oil 22, then through water 24, as shown in FIG. 2. Mathematically the pass through a single large drop of the emulsion may be equivalent to the sum of passes through all the small emulsion droplets taken together. Using basic time, velocity, length and distance relationships the following equations may possibly be derived to result in the measurement of the fraction of water in an emulsion:

The basic time, velocity, length and distance equations:

L=V*T; T=L/V (Equation 1)

The length of the emulsion column is Le shown as reference numeral 26 in FIG. 2:

Le=Lw+Lo (Equation 2)

Where Lw, as shown as reference numeral 28 in FIG. 2, is the length of the water column and Lo, as shown as reference numeral 30 in FIG. 2, is the length of the oil column.

Rearranging Equation 2 above gives:

Lw=Le−Lo (Equation 3)

Water column as a fraction of the emulsion column is Wf:

Wf=Lw/Le (Equation 4)

Substituting for Lw from Equation 3 above gives:

Wf=(Le−Lo)/Le (Equation 5a)

Wf=1−Lo/Le (Equation 5b)

Rearranging Equation 5b gives:

Lo/Le=1−Wf (Equation 6)

Time (Te) for electromagnetic radiation (EMR) propagation through emulsion column Le:

Te=To+Tw (Equation 7)

Where To is time through oil and Tw is time through water. Substituting from Equation 1 for Te, To, & Tw in Equation 7 gives:

Le/Ve=Lo/Vo+Lw/Vw (Equation 8)

Multiply both sides of the above equation by C/Le (where C is the velocity of EMR through vacuum) and rearrange to obtain:

C/Ve=(C/Vo)*(Lo/Le)+(C/Vw)*(Lw/Le) (Equation 9)

Where,

C is the velocity of EMR through vacuum

Le is length of the emulsion column

Lw is length of the water column

Lo is length of the oil column

Ve is velocity of EMR through emulsion

Vo is velocity of EMR through oil

Vw is velocity of EMR through water

The refractive index of a liquid may be defined as the ratio of velocity of EMR through vacuum to that through a given medium: η=C/V. Using this relationship and substituting from Equations 4 and 6 into Equation 9 gives:

ηe=ηo*(1−Wf)+ηw*Wf (Equation 10)

Wf=(72 e−ηo)/(ηw−ηo) (Equation 11)

Where

η—refractive index of medium

ηe—refractive index of emulsion

ηo—refractive index of oil

ηw—refractive index of water

Wf—fraction of water in emulsion

Equation 11 represents the basis for the measurement of the fraction of water existing in an emulsion. Equation 11 may provide a linear relationship between the water content in an emulsion and the refractive index of water. It should be noted that the refractive index is a fundamental property of matter. A measurement of water content in oil from basic principles of physics may be derived from the application of Equation 11. The refractive index of emulsion (ηe) may be determined from measured capacitance and dual point calibration from the following relationship:

η=√(CpF*CIF) (Equation 12)

where

CIF=K*PCF (Equation 13)

CIF—is the composite instrument factor that depends on the type and the installation of the probe

K—is a constant that depends on the specific type and design of the probe

PCF—is a capacitance calibration factor calculated during a dual point integrated probe calibration procedure described later.

CpF=MpF+CCT (Equation 14)

where

CpF—is corrected capacitance specific to each installation

MpF—is the capacitance measured directly from the probe

CCT—is a capacitance correction term calculated during a dual point integrated probe calibration procedure described later

These equations may be applied to various embodiments of the invention, as shown in FIG. 3, wherein the present invention uses the physical property of a liquid's refractive index to detect and measure water content hydrocarbons such as unrefined oils.

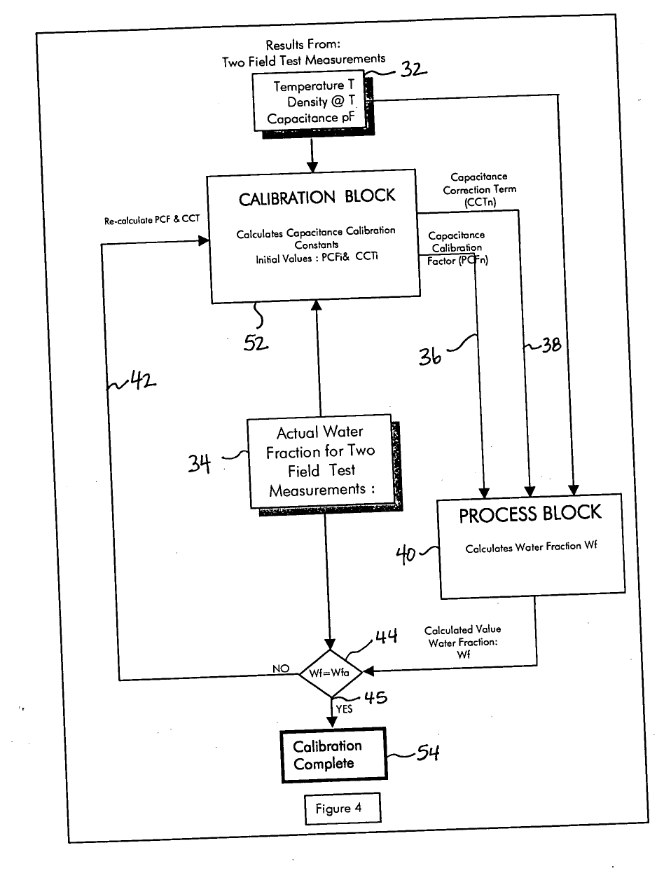

A variety of calibration processes may be applied to the invention. An embodiment of the invention applying an integrated calibration process, as shown in FIG. 4, may utilize: the output of the capacitance device and other field measurements 32; and actual known water contents of separate batches of unrefined oils 34. Each of these elements allows the system to be calibrated in a manner that ensures that it can assess oils having a wide range of densities. Such an approach may use a first batch of oil to derive the capacitance calibration factor value 36 and a second batch of oil to determine the capacitance correction term 38.

FIG. 4 further shows, an iterative measurement procedure that may use the following information: measured capacitance, being the uncorrected capacitance from device output, measured emulsion temperature and measured emulsion density 32; analyzed value of water cut from multiple emulsion samples 34; linear refractive index plot based on published data for refined oils; and Equation 11 to measure the water fraction Wf 40 which is compared to the actual water fraction Wfa obtained at 34. The iterative measurement procedure 42 may ultimately derive 45 a calculated water fraction Wf that is substantially equal to the actual water fraction Wfa. When this occurs, the calibration procedure is complete 54.

The dual point calibration process applies the linear relationship between the density of refined oils and their refractive indices to unrefined oils. In one embodiment of the invention the two batches of oil can be used including one with a lowest density of oil anticipated to be included in the emulsion and one with a highest density of oil anticipated to be included in the emulsion. Each batch would be used one at a time, to calibrate the capacitance device. By selecting oil at the two densities, representing the lower and upper ends of the scale, it may then be possible to calculate water content for all the oils of densities bounded by and outside of the two values selected for calibration. Of course, a single batch or greater than 2 batch calibration can be used as desire.

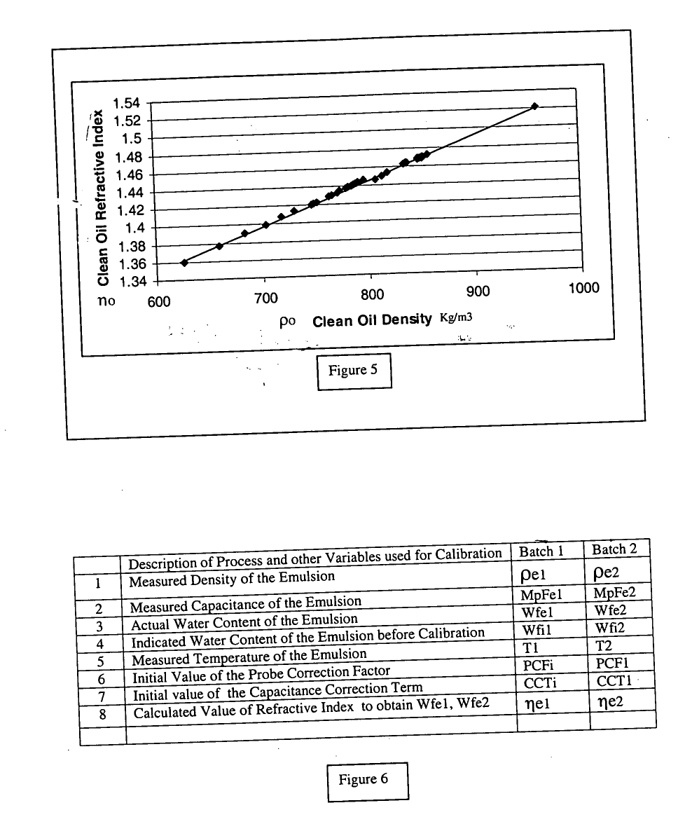

As shown in FIG. 5, the present invention applies readily available data for refined oils, as published in the Gas Processor's Suppliers Association Engineering Data Book and other data sources. FIG. 5 shows, in particular, the linear relationship between the refractive index of oil and its density. Such a linear curve may be generated using curve fit algorithms. Curve fit algorithms may deviate from the ideal density intercept by about 6%, but the application, for example, of integrated dual point calibration may compensate for this and other errors that are inherent and specific to mechanical and electrical installations of a capacitance device.

Errors may also occur if an equation derived from linear non-empirical relationship of refined oils and the linear plot of refractive index versus density, as shown in FIG. 5, are used without reference to the actual refractive index of the emulsion being measured. Therefore, the capacitance device output can be integrated with the linear curve for refined oils and two separate batches of unrefined oils and their water analysis can be applied to the process.

Improper installation of the capacitance device may also cause errors to occur in the signal of the device. A simple and effective method of calibrating and setting up the probe may be achieved by introducing two calibration terms: the capacitance calibration factor 36 and the capacitance correction term 38 as are applied in Equations 13 and 14. Thus, an embodiment of the present invention may calibrate the probe in an integrated manner before using its output to calculate the refractive index values of oil or emulsion.

Process variables and constants for a first and a second batch of emulsion required for calibration of a concentric type of capacitance device are shown in FIG. 6. As would be apparent to a person skilled in the art, other methods of calibration may require variants of the process variables and constants shown in FIG. 6.

Referring again to FIG. 4, a calibration process, as conducted by the computing device 18, utilized in one embodiment of the invention is shown. Measurements gathered from two field tests such as temperature and density, as measured by the apparatus of FIG. 1, may be utilized in this embodiment of the invention. These measurements may be applied to two distinct steps of the calibration process, the calculation of a water fraction 34, as shown in FIG. 3, and the calculation of two capacitance calibration constant initial values 36 and 38. The field test measurements 32 may be transferred directly to each step of the process immediately after the measurements are transferred to the computing device 18.

The field measurements are utilized by the capacitance calculation step 52 to determine two values the capacitance correction term 38 and the capacitance calibration factor 36. The capacitance values 38 and 36 may then be used, along with the field results 32, to calculate a water fraction 40. The two capacitance values 38 and 36 may also be used to determine an actual water fraction 34 according to the two field tests. The water fraction 40 may then be compared to the actual water fraction 34. If the two values match 44, within a proscribed variance, the calibration is complete 54. Should the water fraction values not match, within a proscribed variance, then the calculation 52 of two capacitance calibration constant initial values 36 and 38 will occur again. The whole process will repeat itself iteratively 42 until the water fraction value Wf matches actual water fraction value Wfa.

An embodiment of the invention that incorporates a calibration process with iterative calculation of two calibration parameters, may apply Equations 13 and 14 to the capacitance calibration factor and the capacitance correction term. For the first stage of the calibration procedure, the process and initial values from batch 1, as shown in FIG. 6, may be used to iteratively 42 solve Equation 12. The calculated 52 water fraction Wfi1 44 should ultimately equal the actual water fraction Wfe1 34 of the batch, within accepted deviation limits 44. The initial value PCFi may be set to 1 and the initial value CCTi may be set to 0 for the purposes of an iteration procedure 42. When the iteration converges, the new value PCF1 may be recorded in 18.

The effect of an iteration procedure is shown in FIG. 7. A first curve 58 represents Equation 12 with initial values of PCFi and CCTi. The measured capacitance MpFe1 corresponds to the calculated refractive index ηei which in turn corresponds to water fraction Wfi, as calculated prior to calibration. Second 60 and third 62 curves represent intermediate calculations and a fourth curve 64 represents Equation 12 with value of PCF1 that produces the value of water fraction equal to Wfe1, which is the actual water fraction for batch 1. This embodiment of the present invention as described may represent a complete first stage of the calibration process. The calculation of PCF1 may represent rotation of the first curve 58 around the origin till the desired result is obtained.

As shown in FIG. 6, the value of PCF1 together with the process and initial values from batch 2 may be used to complete the calibration process. In a second step of the calibration process PCF and CCT may be iteratively derived until the water fraction Wfi2 equals the actual water fraction Wfe2, within acceptable deviation limits.

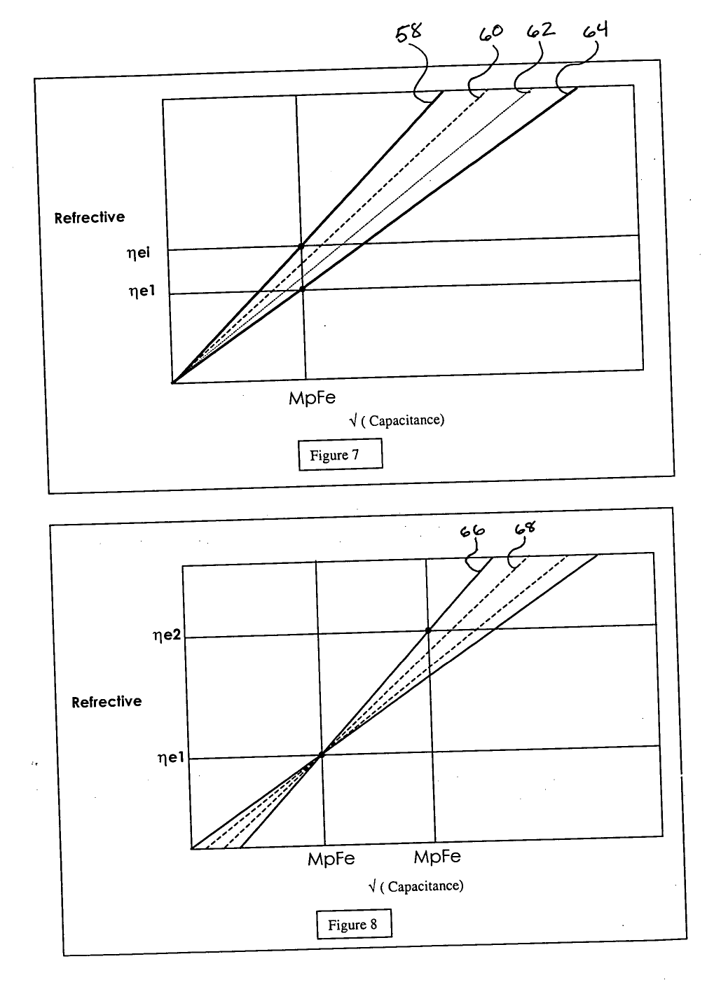

Such an embodiment of the invention is shown in FIG. 8, where a first curve 66 represents Equation 12 based on the initial value of PCF=PCF1. New values of PCF and CCT may be derived to arrive at a second curve 68 which passes through the two points that represent the measurements from the two batches of oil used in the calibration. The derivation of the final values of PCF and CCT may represent rotation of the first curve 66 around the first calibration point until the desired result, being the actual water fraction of batch 2, is obtained. The first curve 66 represents the calibration with the final values of PCF and CCT based on the two batches of unrefined oils.

One embodiment of a process measurement flow is shown in FIG. 3. The capacitance 70, the temperature 72 and the density 74 values of the emulsion flowing through a conduit 10, as shown in FIG. 1, may be measured by the capacitance device 12, the density device 14 and the temperature device 16. A measurement of the density of a brine at 15 deg Celsius may also be made available to the process. The computing device 18 may first use the measured capacitance value 70 together with the capacitance calibration constants 75: the capacitance calibration factor PCF and the capacitance correction term CCT (for example from a process as in FIG. 4), to calculate 76 the raw refractive index ηe 77 for the emulsion using the following equation:

ηe=√(CpFe*CIF)(Equation 12).

The three raw process variables of temperature and density from the measuring devices 14 and 16 and the refractive index ηe may be used to calculate 78 the water fraction according to Equation 11 using the measurement flow of FIG. 8. The following steps may be repeated on an iterative basis till an acceptable convergence in the value for Wf is obtained:

Assume t represents the temperature measured by the temperature device. The first step involves the determination of the refractive index of water ηwt at temperature t from published data in public domain:

ηwt=f(t, ρws) (Equation 15a)

The density of water ρwt at temperature t, and the volume correction factor for water (VCFw) from published data in public domain can then be derived:

VCFw=f(ρws) (Equation 15b)

ρw,t=VCFw*ρws (Equation 15c)

The second step 82 is to estimate the refractive index of oil ηot at temperature t, starting with initial value of Wft=0, from the measured refractive index of emulsion ηet and through iterations to estimate the density pot of clean oil at temperature t:

ρot=(ρet−Wft*ρwt)/(1−Wft) (Equation 16)

ηot=f(ρot) (Equation 17)

Equation 17 derives the linear plot 84, as shown in FIG. 3. From Equations 12, 15a and 17 the fraction of water in emulsion from Equation 11 at temperature t may be estimated:

Wft=f(ηet, ηot, ηwt) (Equation 18)

The current water fraction value 88 may be compared 90 to the previous water fraction value 92. Each subsequent current water fraction value is saved to the computing device 18 memory to be utilized as the next previous water fraction value. If the current 88 and previous 92 water fraction values do not match within an acceptable variance, steps 82, 84, 78 and 90 may be repeated iteratively 93 until step 90 achieves an acceptable result.

Equations 11, 16 and 17 form the main part of the process. When the iteration converges to specified limits, the last value for water fraction, Wft, at a measured temperature is obtained 92. Since all calculations of measured volumes of the emulsion must be at reference conditions, normally being 15 deg Celsius, the third step 94 converts the water fraction at Wft to Wfr at a reference temperature of 15 deg Celsius.

The third step 94 derives the water fraction at reference conditions of 15 deg Celsius. Wfr=f(VCFo, VCFw, Wft) (Equation 18), where VCFo is the volume correction factor of oil, as calculated from the American Petroleum Institute Standard.

This provides an output 96 water fraction Wf for the emulsion being analysed.

Naturally, the invention is not limited to the embodiments described and variants can be made thereto while falling within the ambit of the invention as defined by the claims.

We claim:

1. An apparatus for measuring the content of water in an emulsion including hydrocarbons and water, the apparatus comprising:
a conduit through which the emulsion may flow from a first point to a second point;
at least one measurement device selected to measure density and capacitance of the emulsion within the conduit to generate a density value and a capacitance value; and
a computing device capable of receiving the density value and the capacitance value from the at least one measurement device and for the purpose of determining the content of water in the emulsion through the application of a refractive index.

2. An apparatus for measuring the content of water according to claim 1 wherein the emulsion is a mixture of unrefined oil and water.

3. An apparatus for measuring the content of water according to claim 1 wherein the at least one measurement device further is selected to measure temperature of the emulsion within the conduit to generate a temperature value.

4. An apparatus for measuring the content of water in hydrocarbons according to claim 1 wherein the conduit is a pipe for conveying oil at a storage facility.

5. An apparatus for measuring the content of water in hydrocarbons according to claim 4 wherein the pipe extends between a truck and a holding tank whereby the emulsion is off-loaded from the truck into the holding tank.

6. An apparatus for measuring the content of water in hydrocarbons according to claim 1 wherein the at least one measurement device is a capacitance device capable of deriving a capacitance value.

7. A method for measuring the content of water in an emulsion including hydrocarbons and water, the method comprising:
providing at least one measurement device for obtaining a density value and a capacitance value from the emulsion;
providing a computing device capable of receiving the density value and the capacitance value from the at least one measurement device; and
using the density value and the capacitance value to determine: a refractive index value of the emulsion; at least one calibration value; and at least one water content value of the emulsion.

8. The method for measuring the content of water according to claim 7 wherein the emulsion is a mixture of unrefined oil and water.

9. The method for measuring the content of water according to claim 7 wherein the at least one measuring device is useful for obtaining a temperature value from the emulsion and the method further comprises using the temperature value to determine at least one calibration value and at least one water content value of the emulsion.

10. The method for measuring the content of water according to claim 9 wherein the computing device accepts a first set of temperature, density and capacitance values and a second set of temperature, density and capacitance values, each set of values being derived from two individual measurements from the emulsion by the at least one measurement device and utilizes both sets of values to determine the refractive index value, the at least one calibration value and the at least one water content value.

11. The method for measuring the content of water according to claim 7 wherein the computing device applies an iterative process to its determination of at least one of the at least one calibration value and the at least one water content value of the emulsion.

12. The method for measuring the content of water according to claim 7 wherein the emulsion is being handled at a hydrocarbon storage facility.

13. The method for measuring the content of water according to claim 7 wherein the density value and the capacitance value are obtained from the emulsion as it is being conveyed through a pipe.

14. A method for measuring the content of water in an emulsion including hydrocarbons and water, the method comprising:
obtaining a density value and a capacitance value from the emulsion;
and using the density value and the capacitance value to determine: a refractive index value of the emulsion; at least one device calibration value; and at least one water content value of the emulsion.

15. The method for measuring the content of water according to claim 14 wherein the emulsion is a mixture of unrefined oil and water.

16. The method for measuring the content of water according to claim 14, the method further comprising obtaining a temperature value from the emulsion and using the temperature value to determine the at least one calibration value and the at least one water content value of the emulsion.

17. The method for measuring the content of water according to claim 16 wherein the step of obtaining includes obtaining a first set of temperature, density and capacitance values and a second set of temperature, density and capacitance values, each set of values being derived from two individual measurements from the emulsion and wherein both sets of values are used to determine the refractive index value, the at least one calibration value and the at least one water content value.

18. The method for measuring the content of water according to claim 14 wherein the computing device applies an iterative process to its determination of at least one of the at least one calibration value and the at least one water content value of the emulsion.

19. The method for measuring the content of water according to claim 14 wherein the emulsion is being handled at a hydrocarbon storage facility.

20. The method for measuring the content of water according to claim 14 wherein the density value and the capacitance value are obtained from the emulsion as it is being conveyed through a pipe.
